(19) United States
(12) Patent Application Publication
(10) Pub. No.: US 2015/0342737 A1
(43) Pub. Date: Dec. 3, 2015

(54) MITRAL AND VENTRICULAR GEOMETRY RESTORATION SYSTEMS AND METHODS

(71) Applicants: Brian A. BIANCUCCI, Chelsea, MI (US); Michael J. O'DONNELL, Ann Arbor, MI (US)

(72) Inventors: Brian A. BIANCUCCI, Chelsea, MI (US); Michael J. O'DONNELL, Ann Arbor, MI (US)

(21) Appl. No.: 14/727,401

(22) Filed: Jun. 1, 2015

Related U.S. Application Data
(60) Provisional application No. 62/005,363, filed on May 30, 2014.

(51) Int. Cl. A61F 2/24

(52) U.S. Cl. CPC A61F 2/2442; A61F 2/2487; A61F 2/2481; A61F 2/2466; A61F 2220/0008

(57) ABSTRACT

Apparatuses and method of using them, for restoring and reshaping the mitral valve annulus and reduce or restore the lengthwise geometry of the heart. These apparatuses may include two or more support chords each having an anchor at the distal end for connecting to a valve annulus, and an elongate length. The support chords may be held within a thin delivery cannula. Also include a bendable/conformable apical cradles to which the proximal end of the chords may be attached. The attachment to the cradle or sling member may be adjustable, so that the length of the chords may be adjusted from outside of the heart later. Attaching the support chords to the valve annuls and anchoring on either side of the apex region of the heart may reshape both the mitral valve and the ventricle length.

### CROSS REFERENCE TO RELATED APPLICATION

This application claims priority to U.S. Provisional Application No. 62/005,363, filed May 30, 2014, titled “MITRAL AND VENTRICULAR GEOMETRY RESTORATION SYSTEM,” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This invention relates generally to systems and methods for treating cardiac dysfunction, and more specifically to systems and methods for repairing and/or reshaping the heart valves and heart chambers.

### BACKGROUND

The mitral valve apparatus, in addition to controlling flow between the left atrium and left ventricle, is an important component of the structural integrity of the left ventricle. The valve leaflets, chordae, papillary muscles and left ventricular wall all work in unison to provide proper force balance and stress distribution in the heart. The mechanical support provided by these structures is directly related to their geometry and three dimensional positioning relative to each other. The chordae in particular act as tensioning members for the valve and ventricle. While the primary chordae connect to the leaflet edges and prevent prolapse, the secondary or basal chordae connect nearer to the annulus, and support most of the tensile load.

In certain myocardial disease states, the ventricle begins to dilate, causing the posterior wall of the left ventricle and papillary muscles to move away from the mitral annulus. The resulting abnormal lengthening of the mitral apparatus and dilation of the annulus can lead to regurgitation, which exacerbates cardiac dysfunction. Dilation can also cause stress to be transferred from the thicker basal chords to the primary chords, which may accelerate structural failure of the valve. Cardiac function is also compromised by overall lengthening of the ventricle, because dynamic shortening (i.e., movement of the mitral annulus towards the apex) is critical for efficient pumping.

In some patients, mitral regurgitation can be treated with surgical repair or replacement of the valve. An annuloplasty ring may be implanted to treat the radial dilation of the valve annulus. However in many patients, even after annuloplasty ring implantation, some valve regurgitation may persist due to the unresolved lengthwise displacement (tethering) of the papillary muscles and ventricular wall. In addition, there are many patients who are not suitable candidates for surgery. Annuloplasty alone may not address tethering and therefore recurrence of mitral regurgitation.

Non-surgical approaches for reshaping a diseased left ventricle have typically involved external wraps that are intended to reduce the volume of the ventricle by compression. These devices create a general or global inward force, but do not allow for targeted geometric adjustments or reshaping and do not necessarily work along the long axis of the ventricle.

Other non-surgical technologies (or minimally invasive) have attempted to deliver devices into the heart by direct puncture of the ventricular wall. However, a separate procedure or technology may be required to repair the puncture site if the puncture is created by a device of a certain size.

Thus, there is a need for apparatuses (devices and systems) and methods to restore cardiac geometry. In particular, what is needed are apparatuses and methods for the targeted, comprehensive reshaping of the mitral valve and the ventricle, which may restore structural support between the mitral annulus and the papillary muscles, reduce the A-P dimension, and reduce leaflet tethering, and reduce leaflet tethering and apical displacement of papillaries. In particular, a simple and quick procedure using minimal implanted materials that has a small insertion profile (e.g., having a 1.7 mm diameter catheter diameter or smaller to avoid leaving a permanent hole or causing persistent bleeding) would be beneficial. Described herein are systems and methods that may meet these criterions and address the needs discussed above.

### SUMMARY OF THE DISCLOSURE

In general, described herein are apparatuses (e.g., devices and systems), and method of using them, for restoring and reshaping the mitral valve annulus and reduce or restore the lengthwise geometry of the heart, and particularly the ventricle. These apparatuses may include two or more chords (e.g., support chord, tethers, strings, tendons, fibers, sutures, wires, cords, etc., which may function as artificial chordae tendineae) having an anchor at each distal end, and an elongate length. The chords may be held within a thin (e.g., 5 Fr or less) delivery cannula. The anchor is typically configured to anchor the chord into the annulus of the mitral valve after passing through the wall of the ventricle lateral to the ventricle apex. These apparatuses may also include a bendable/conformable apical cradle or sling member to which the proximal end of the chords may be attached. The attachment to the cradle or sling member may be adjustable, so that the length of the chords may be adjusted from outside of the heart later (e.g., hours, days, weeks, years) after the apparatus has been implanted. This may allow for minimally invasive adjustment of the apparatus. For example, the attachment to or through the sling may be adjustable to tighten or loosen the support chord.

The apparatus may be configured as a system including the support chords, apical cradle and any other components useful or helpful for connecting and/or adjusting the device, such as a sheath and/or needle for inserting and attaching the support chords, a dilator, and securement devices (e.g., adjustable chord attachment mechanism).

For example, a system for reshaping the geometry of a diseased heart may include: a cradle for supporting an apical portion of the heart, the cradle having a central portion for supporting the apical portion of the heart, a first end for supporting a first side of the heart, and a second end for supporting a second side of the heart, the cradle having a first reinforced structure disposed on the first end and a second reinforced structure disposed on the second end, wherein the cradle is made from a flexible material; a first support chord having a distal end and a proximal end, the distal end of the first support chord comprising a first anchor, the proximal end of the first support chord configured to be secured to the first reinforced structure; and a second support chord having a distal end and a proximal end, the distal end of the second support chord comprising a second anchor, the proximal end of the second support chord configured to be secured to the second reinforced structure.

The first anchor may be configured to be oriented parallel to the first support chord during insertion into the heart and substantially perpendicular to the first support chord after implantation in the heart. Both the first anchor and the second anchor may have a cross-sectional profile sized for allowing passage of the first anchor and the second anchor through an 18 Gauge or smaller needle or a 5 French or smaller sheath (e.g., they may be smaller than 1.7 mm diameter), and they may be any appropriate length and shape for anchoring around the atrial side of an annulus. For example, the first anchor and the second anchor have a length between about 2 to 10 mm. The anchors may be rigid, elongate tubular members through which the first support chord is passed. For example, the tubular member may have a cutout or slot extending from both ends of the tubular member. The tubular member may have two or more holes configured to receive the first support chord.

Any of the systems described herein may include a delivery sheath or needle configured receive the first support chord and the first anchor.

The support chord may be made of any appropriate material. In general, this material may have a very low creep, so that even over an extended time of implantation the length does not change significantly. The support chord may be made of a flexible material or alternatively, a rigid material. The support chords may be made from a shape memory wire or tube having a distal end that is shape set to form the first anchor (or may be attached to a shape memory anchor). For example, the support chord may be a polymeric material, such as a prolene suture. In some variations the support chord is a monofilament; in some variations the support chord is a woven materials.

In general, the apical cradle may be formed of a material that is sufficiently flexible or conforming so that it conforms to the curved outer (epicardial) surface of the apex of the heart. The apical cradle may be pre-shaped (e.g., having a U- or C-shape), or it may be sufficiently shapeless over at least a middle region (between distal and proximal chord attachment regions) to conform to the outer surface of the heart. For example, the apical cradle may be made of a fabric or membrane. In some variations, the cradle may have one or more bands or struts of shape memory material that are configured to adopt a predetermined shape after insertion (e.g., a C- or U-shape); the predetermined shape may correspond to an apical portion of the heart.

The reinforced structure of the apical cradle may comprise a pad, such as a pad having a layer of semi-rigid material and a layer of compliant material.

Any of the apparatuses described herein may include a securement device configured to secure a support cord a reinforced structure of the apical cradle, wherein the securement device is configured to adjust the length of the support cord after it has been secured to the reinforcement structure. The securement device may be integrated into (e.g., part of) the apical cradle, or it may be a separate element. The securement device may be fastened to the apical cradle before or after attaching a support cord. For example, a securement device may be a spring-loaded or snap-fit clip. In some variations, a securement device may include a pair of vertical halves of a cylinder that are surround by a rotatable housing configured to compress the pair of vertical halves together. In some variations, a securement device may include a first rigid plate with a first hole for receiving the first support cord and a second rigid plate with a second hole for receiving the first support cord, wherein the first hole and second hole are offset from each other. A securement device may include a male threaded component and a female thread component. As mentioned, in some variations the securement device is integrated into the apical cradle. For example, a cradle may include a pair of rotatable reel mechanisms configured to secure and tighten the support chord(s).

In any of the apparatuses described herein a cinching device may be included for connecting the support chords within the ventricle of the heart. For example a cinching device may be configured to be slidably disposed over both the first support chord and the second support chord in order to reduce the distance between the first support chord to the second support chord.

The securement device may have one or more holes or slots for receiving the first support chord. Alternatively or additionally, the reinforced structures at the ends of the cradle may include holes, channels, or guides for receiving the support chord(s). In general the cradle may be flat (e.g., may have a length that is greater than its width, and a thickness that is much less than the length and width). The reinforced structures at either ends may also include, or may include attachment sites for holding, a securement device.

In general, the procedure for restoring and/or reshaping a mitral valve annulus and/or the length of the heart may generally include insertion of the distal end of a chord, which may be held in a sheath and/or trocar, through the patient's papillary and mitral valve annulus. Thereafter, the trocar may be removed, and the anchor (which may be referred to as an annulus anchor) at the distal end of the support chord may deployed from the atrial side of the annuls. The proximal end of the support chord passes through the wall of the ventricle, and may be attached through an apical cradle that wraps at least partially around the outside (epicardial region) of the apex. Support chords may be placed symmetrically around the annulus (e.g., two support chords on opposite sides, or three triangulated support chords, etc.). Within the ventricle, the support chords may be tethered together, or they may cross each other, or they may not cross each other.

For example, a method for reshaping the geometry of a diseased heart may include: inserting a first support chord through a first apical portion of the heart; anchoring the first support chord to a first location on the mitral annulus; inserting a second support chord through a second apical portion of the heart; anchoring the second support chord to a second location on the mitral annulus; placing a cradle against the apical portion of the heart; securing the first support chord and the second support chord to the cradle; and tensioning the first support chord and the second support chord to secure the cradle against the apical portion of the heart.

Any of these methods may also include: inserting a sheath and trocar through the first apical portion of the heart to the first location on the mitral annulus; inserting the sheath and trocar through the mitral annulus and into the left atrium at the first location on the mitral annulus; removing the trocar from the sheath; and inserting the first support chord through the sheath.

Tensioning the first support chord and the second support chord may reduce the size of the mitral annulus and shortens the length of the left ventricle of the heart.

In general the support chords may be positioned around (e.g., symmetrically around) the mitral annulus. For example, the first location on the mitral annulus may be opposite the second location on the mitral annulus. The first location on the mitral annulus may be located on an anterior portion of the mitral annulus and the second location on the mitral annulus is located on a posterior portion of the mitral annulus.

Any of these methods may also include inserting the first support chord through papillary muscles in the left ventricle of the heart. The first support cord and the second support chord may be inserted in a crossing configuration. The first support cord and the second support chord may be inserted in a non-crossing configuration. Any of the methods described herein may also include tensioning the first support chord and the second support chord laterally inwards by cinching the first support chord and the second support chord together. For example, the first support chord may be not parallel to the second support chord after insertion into the heart.

Any of these methods described herein may also include tensioning a support chord and under echocardiogram visualization until a desired reduction in mitral insufficiency is observed or a desired shortening in the length of the ventricle is observed or a desired change in shape of the mitral annulus is observed.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is an example of a system for reshaping the geometry of a diseased heart, including a needle, pair of sheaths, and a hypotube loaded with a support chord with an anchor.

FIG. 2 shows a variety of different needles and sheaths that may be used to insert the support chords.

FIG. 3 shows a support chord (suture with anchor), sheath and needle, and dilator that may be included with any of the apparatuses described.

FIGS. 4A and 4B illustrates the operation of a first example of an anchor on a support chord, as described herein in a deployed 401 (FIG. 4B) and undeployed 403 (FIG. 4A) state.

FIG. 4C illustrates a variation of the anchor including a pledget that may distribute the force of the anchor on the tissue.

FIG. 5 shows one example of an apical cradle having a proximal and distal reinforced region.

FIG. 6 is an example illustrating introduction of a sheath and needle holding a support chord from a ventricular apical region through the ventricle and into the underside of the mitral annuls.

FIG. 7 is an enlarged view of the sheath and needle penetrating through the mitral annulus and into the left atrium.

FIG. 8 illustrates an implanted anchor and support cord from FIG. 7 around the mitral annuls.

FIGS. 9A and 9B illustrate an anchor of a support chord in an undeployed (FIG. 9A) and deployed (FIG. 9B).

FIGS. 10A and 10B illustrates views of alternative variations of anchors as described herein.

FIG. 11 illustrates the use of crossing patterns for chords within the ventricle. Crossing (or otherwise tethering them together) within the heart may distribute the forces acting through the support cords and on the heart.

FIG. 12 illustrates another variation using a cinching device to cinch the middle region of the support cords within the ventricle to reduce the distance and/or independence of the support cords after being implanted. The cinching device may also help distribute the forces acting at the anchors and/or attachment site.

FIG. 13A shows one variation of an apical cradle having a pair of reinforced regions. Attached to apex of the heart.

FIG. 13B is an enlarged view of the implanted cradle.

FIGS. 14A and 14B illustrate another variation of a cradle from a first perspective view and a second perspective view.

FIG. 15A illustrates one variation of a cradle device for an apparatus for reshaping the geometry of a diseased heart.

FIG. 15B illustrates the cradle of FIG. 15A applied over a test model of the distal (apical) end of the heart.

FIG. 16A shows one example of a reinforced region of an apparatus for reshaping the geometry of a diseased heart.

FIG. 16B shows the reinforced region of the cradle flexing.

FIG. 17A illustrates one example of an apical cradle; in this example, the cradle includes a pair of integrated reel mechanisms that maybe tightened (e.g., using a screw) to tighten/loosen the support suture that is attached thereto.

FIG. 17B is a close-up view of the securement device of FIG. 17A.

Similarly, FIG. 18A is a perspective view of a securement device that may be used to periodically and manually adjust the length and/or tension of an implanted support chord, and

FIG. 18B illustrates a section through the securement device of FIG. 18A.

FIGS. 19A and 19B illustrate different securement devices or portions of securement device that may be used to retain and release a support chord.

FIGS. 19C and 19D illustrate the operation of one variation of a securement device.

FIG. 20A is an example of a procedure for forming and connecting a support chord to an apical cradle.

FIG. 20B is a slightly enlarged view of the cradle, support chord, anchoring securement device and a portion of a suture.

FIG. 21 illustrates one example of a snap-fit chordal securement device for connecting to a support chord to a cradle so that it can be adjusted.

FIG. 22 illustrates the interaction between a transeptal catheter for use in annulus identification and anchoring.

FIG. 23 is an enlarged view showing attachment of a support chord anchor to the apical side of an annuls; in this example, a separate anchor couples beneath the annuls.

FIGS. 24A-24D illustrate the closure of a left atrial appendage.

### DETAILED DESCRIPTION

In general, described herein are apparatuses and methods for restoring and reshaping the mitral valve annulus and reduce or restore the lengthwise geometry of the [symbol] . As used herein an apparatus may be a device or system (e.g., interrelate collection of components) that can allow installation of two or more supports chords through the ventricle to be anchored at one end at the annuls of the mitral valve, and at the other end to a cradle over the apex of the outside of the heart.

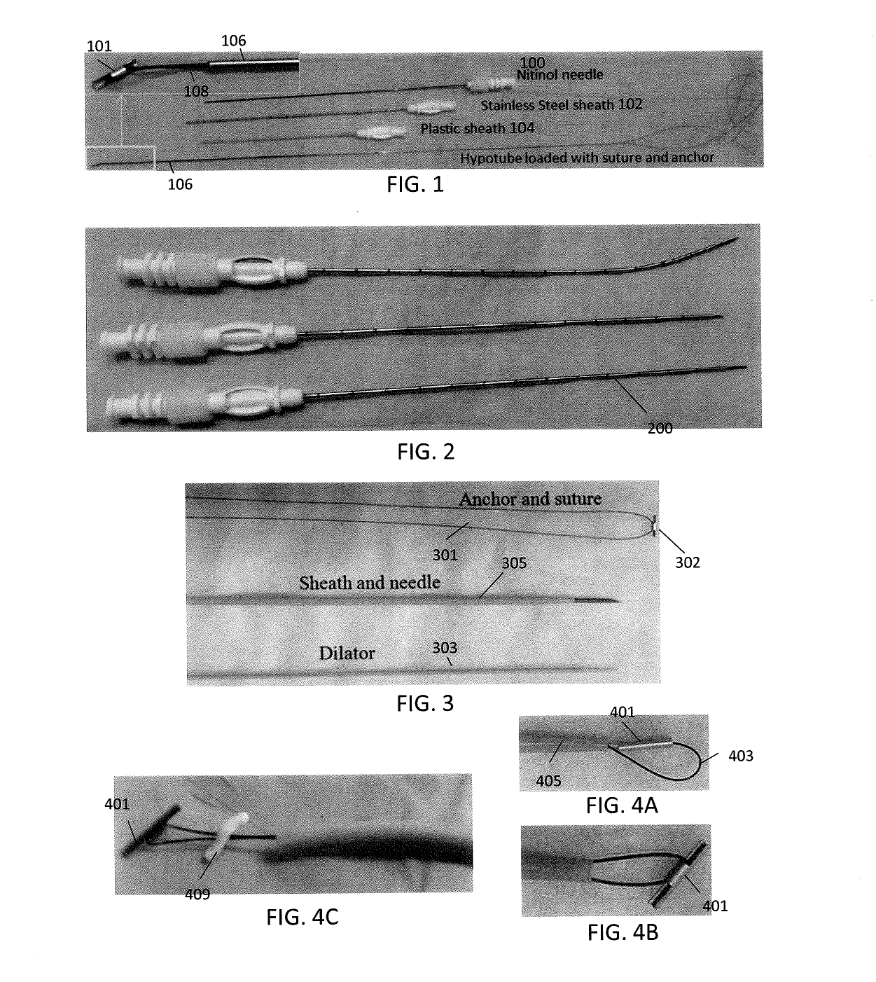

For example, FIG. 1 shows one example of a system for remodeling the mitral valve annulus and length of ventricle. Specifically, FIGS. 1 and 2 illustrate various tools that can be used to perform the procedure. FIG. 1 shows an embodiment of the solid needle 100, which can be made of a metal such as Nitinol or stainless steel. The solid needle 100 can be inserted into a sheath, which can be a stainless steel sheath 102 or a plastic sheath 104, which can facilitate insertion of the sheath through the heart tissue. A hypotube 106 that can fit into the lumen of the sheath 102, 104 can be loaded with the support chord 108 and 110. FIG. 2 illustrates various embodiments of the metal sheaths with depth markings along the length of the sheath.

The support chords can be made of suture, fabric or similar flexible materials or can be made from more rigid materials such as metal wire. In some embodiments, the support chords are prolene suture. The support chord maybe monofilaments, or braded or woven materials. FIG. 3 shows an anchor and suture 301, as well as a sheath and needle 305 and dilator 303. The suture includes an anchor region 302. A close of this anchor region may be seen in FIGS. 4A and 4B, showing an anchor attached to the distal end of a bight (e.g., loop) of support chord. Note that the support chord may be a loop, or it may be a single length of material. When the support chord is a loop of material, the term distal end may refer to the distal end of the loop, e.g., the region where it is or can be doubled back over itself (changing direction), as shown in FIG. 3, top.

In FIG. 4A the distal end of the (loop of) support chord 403 is extending from within a needle or sheath 405, and the anchor 401 is in the undeployed state, held parallel (in-line) with the length of the support chord. Once the distal end region of the support chord 403 has been passed through the tissue, e.g., through the annulus of the mitral valve, the anchor 401 may be deployed as shown in FIG. 4B, so that the anchor is perpendicular to the direction in which the support chord extends. In some variations, the support chord includes a washer or other pledget 409 at the distal end region, which may also help distribute the anchoring force.

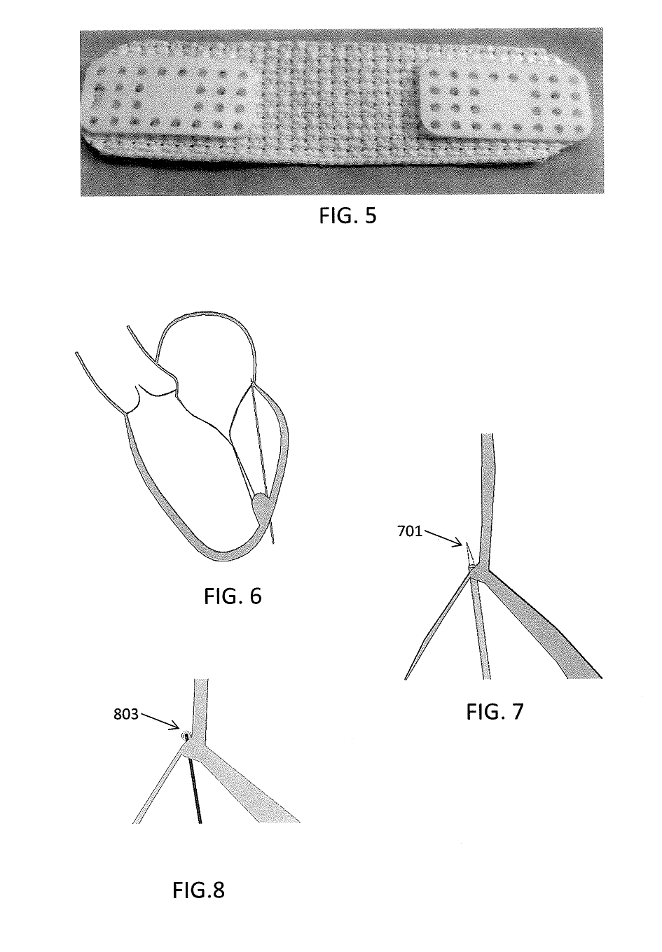

FIG. 5 illustrates one example of an apical cradle that may be used as part of the apparatuses described herein. In general, the apical cradle is flat, and can conform to, or is pre-shaped to conform to, the outside of the apex of the heart. In FIG. 5, for example the elongate, flat apical sling is formed of a fabric material that is generally compliant, but has two reinforced end regions formed of harder (higher durometer) plastic. These end regions include passages or guides through which the support chords may pass and later anchor against; the somewhat rigid end regions may thus distribute the force applied by the support chords across a larger area, preventing further damage to the heart.

As discussed above, the present invention may be an apparatus (e.g., a device or system) and method for placing and anchoring an end of one or more artificial chords through the mitral annulus and securing the opposing end(s) outside the ventricle along with a cradling device to support the ventricle, in order to induce a beneficial shape change and thus reduce the stress on the mitral apparatus and ventricular wall.

For example, procedure steps for this method can be summarized as follows. A small sheath may be introduced into the left ventricle with the aid of a solid needle disposed in the sheath, passed through the posterior papillary muscle and, under image guidance, directed to the underside of the mitral annulus, just adjacent to the hinge point of the leaflet of the mitral valve. This is illustrated in FIGS. 6-8. The sheath and needle are pushed through the annulus to the atrial side of the mitral valve. The needle is removed and an anchor/chord assembly is pushed through the sheath into the left atrium. The anchor is deployed and secured against the atrial surface of the annulus. The sheath is removed, leaving the chord to pass from the anchor, through the LV and papillary muscles to the exterior of the heart. At least one other or multiple anchor/chord assemblies may be similarly placed at other annulus locations. The externalized chords are then each passed through an apical cradle or sling and secured under tension.

The distal end of the chord typically includes an anchor, which is configured to have a low cross-sectional profile for insertion but which has substantial length to provide adequate surface area for anchoring. The anchor can be oriented vertically (i.e., parallel to the axis of the delivery system catheter and/or the support chord material) for insertion and then adjusted to be horizontal and substantially perpendicular to the chord in its implanted position. The adjustment from vertical to horizontal can occur through shape-setting the anchor/chord connection, by spring force, or by manually tensioning or manipulating one end of the chord.

The chord and anchor are configured to fit through a delivery device of a size that is known to cause negligible trauma to the heart (e.g., 18 Gauge or less needle, 4 or 5 French or less sheath) and that precludes the need for closure or sealing procedures to the heart wall. The sheath may be straight or have a curvature. The delivery sheath may be inserted into the left ventricle from the apical aspect, either at the location of a papillary location or an adjacent region. A sharp penetrating tool, such as a solid needle, may be housed inside the sheath and used to penetrate the muscle. Using standard imaging techniques, the delivery sheath and penetrating tool are then advanced to a position underneath the valve annulus, but not in contact with a valve leaflet, as illustrated in FIG. 6. The penetrating tool 701 is then pushed forward into the atrial chamber, as shown in FIG. 7.

The penetrating tool is then removed, leaving the delivery sheath and providing access between the outside of the heart and the left atrium. The chord and anchor 803 can be delivered through this sheath. The anchor 803 is deployed into the left atrium, transitioned to the horizontal orientation, and then pulled back to rest against the atrial side of the mitral annulus as shown in FIG. 8. In this way, anchoring does not depend on embedding into the tissue itself.

Once the anchor is secured, the delivery sheath is removed, leaving the chord(s) exiting the heart muscle at puncture site. The chord(s) can then be tensioned by the operator to induce a desired amount of shape change in the heart.

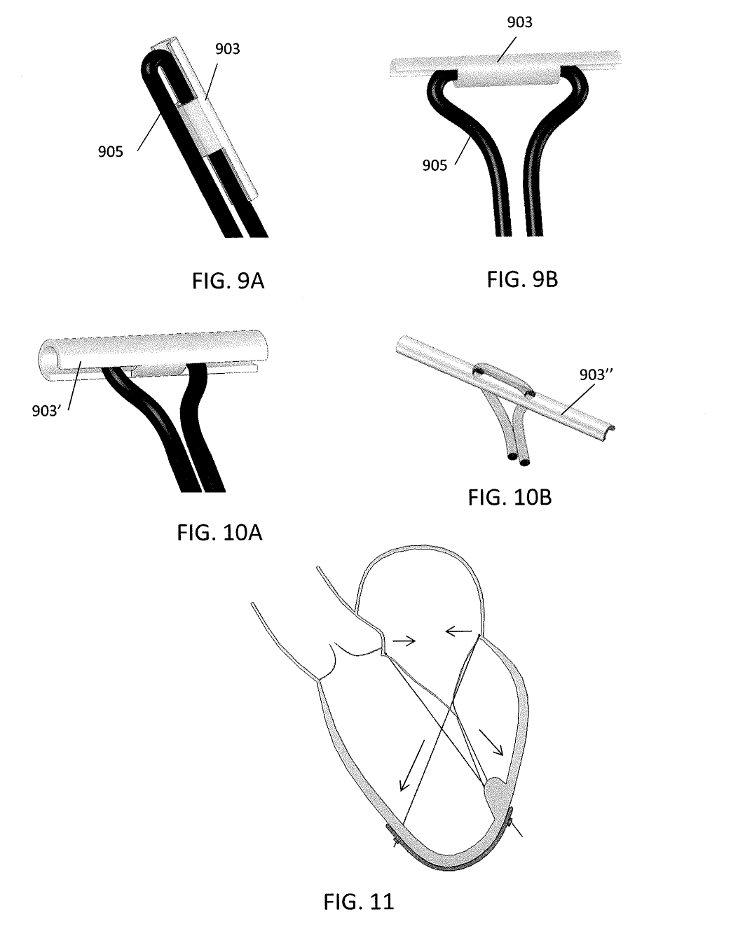

In one embodiment of the chord 905 and anchor, the anchor 903 is made from a rigid tube with half of the tube wall cut away for some length at either end. A length of uncut tube remains in the middle. As shown in FIGS. 9A-9B, the chord material is passed through this tube. The chord may be secured to the anchoring tube by crimping or bonding, or left unsecured. Other anchor designs include a tube with slot cut ends rather than 180 degree cut-away, and a pair of holes through which to run the chord. FIGS. 10A-10B show alternative illustrations of anchors 903′, 903″ similar to the anchor shown in FIGS. 9A-9B.

The anchor will exit the delivery sheath in the vertical orientation but can be reoriented horizontally by pulling on one end of tensioning chord from the proximal end or by spring force. This process can be reversed if it is necessary to return the anchor into the sheath.

Other anchor-chord embodiments include: the chord may be made from a shape memory wire in which the distal end has a hook, curled loop (“pig tail”), or 90 degree bend shape set into the wire. The shaped end can be straightened for delivery through the sheath, and then released in the left atrium for anchoring. The distal anchor may also be an expandable shape memory element. The distal anchor may also be an inflatable element such as a small balloon, and the balloon can be filled with a compound or combination of compounds which harden after injection.

The invention allows for the placement of multiple anchors at key locations (e.g., two anchors on opposing sides of the mitral annulus). The delivery of two anchors may be performed so as to create “crisscross” pattern in the chords as shown in FIG. 11. The chords connected to these anchors will not be parallel to each other, but each will be angled toward its puncture site in the ventricle. As a result, creating longitudinal tension on the chords will also create some horizontal force and allow the anchors to move toward each other. This movement will be beneficial for reducing the diameter of a dilated annulus.

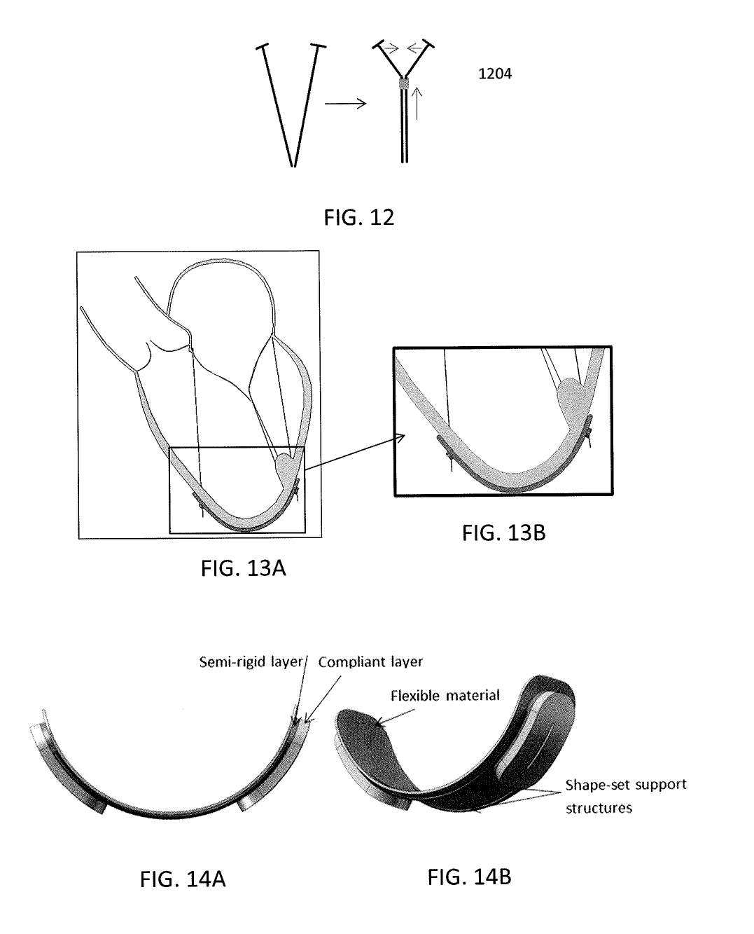

Another mechanism for moving anchors closer together is to slide a cinching device over two or more pair of chords and advance the cinch towards the anchors shown in FIG. 12. The cinching device 1204 can be any device which constrains the chords together at a point, such as a tube. The cinching device may be secured in place by crimping, ultrasonic welding, compression, or bonding.

### Apical Cradle Support:

Before securing the chords, the proximal ends are passed through a flexible band of material that is of a width and length to span all external anchoring points and provide support to the apical aspect of the heart (“apical cradle”), as shown in FIGS. 13A-13B. In the area where the sutures pass through, additional material or pads may be included in the cradle. The pads may include multiple layers of material. A semi-rigid material may be used to provide structural support, while a soft, rubbery material may be used to introduce compliance and reduce peak stresses in the system.

FIGS. 14A and 14B illustrate another variation of an apical cradle. The cradle itself may be made from a soft, flexible material (e.g., fabric or silicone) that allows insertion into the chest cavity through small incisions. The cradle may alternatively include one or more bands of shape set material (e.g., Nitinol) that allows the cradle to take a predetermined shaped after insertion.

The cradle concept is an improvement to local anchoring of the proximal chord ends because it offers better stress distribution, aids in the geometric reshaping of the left ventricle (e.g., moving dilated papillary muscles toward each other) and provides for increased support for the ventricular wall in conjunction with the mitral annulus anchors.

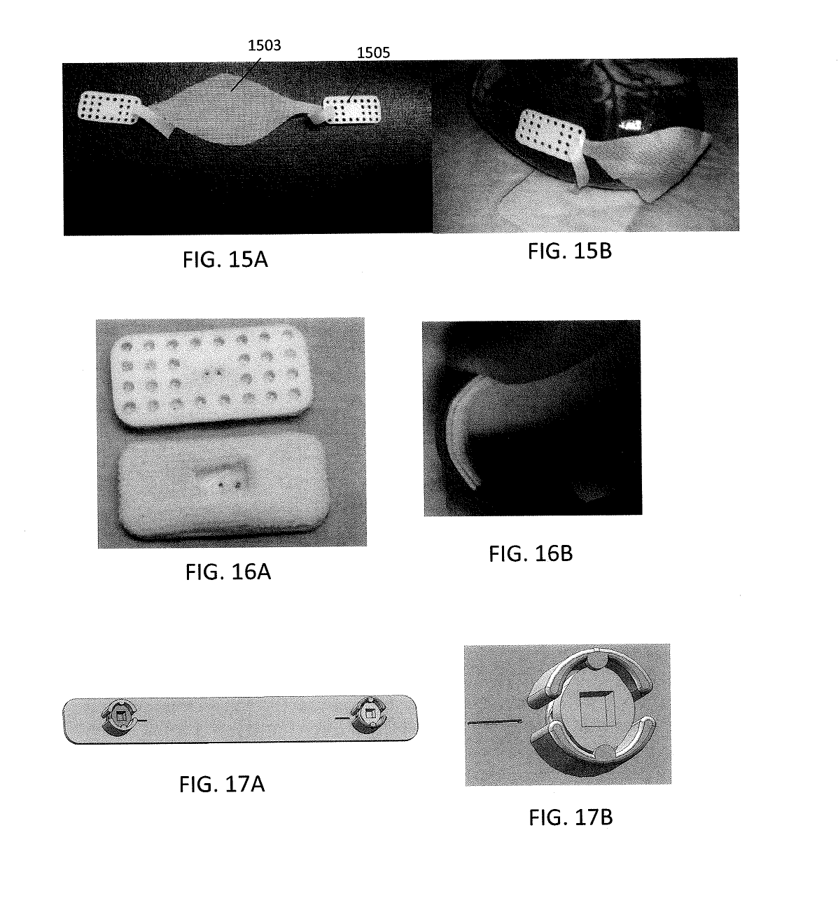

FIGS. 15A and 15B show another example of an apical cradle 1503 having two rigid regions 1505 (reinforced regions) at the distal ends. In this example, the distal ends extend from a flexible intermediate region that does not underlie the rigid region. Any of the cradles described herein may include reinforced regions (also referred to as rigid regions) that may spread the force of the attachment of the thin support chord out across a larger surface area. For example, FIG. 16A shows an example of a reinforced (rigid or stiffer) region that includes a plurality of channels through the relatively stiffer body to allow passage of a needle or cannula for placement of a support chord. Although relatively stiffer than the connection region of the cradle, a reinforced region may also be flexibly and/or may be pre-formed into a curved shape to better conform to the side of the heart, as illustrated in FIG. 16B.

### Chord Tensioning and Securement:

After the chords have passed through the cradle they are tensioned and locked in place so as to maintain the tension. This may be achieved by manually tensioning the sutures and tying a knot. However, manual access for knotting may be limited due to the small incisions that will be used.

Tensioning may instead be accomplished by pushing or sliding a securement device down the length of the suture with a tool and then, while under tension, deploying the device. The securement device may be a spring-loaded clip or snap-fit clip currently used to hold sutures fast in lieu of knot tying.

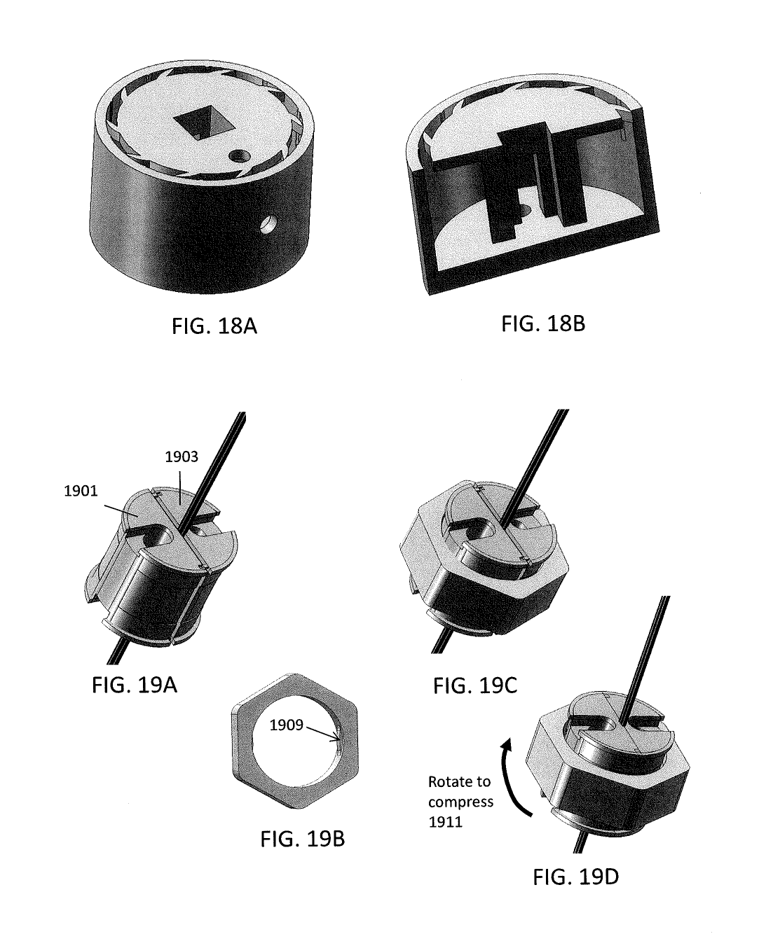

Another method for tensioning the suture is to wrap it around a rotatable spool, or reel. Such a reel could be incorporated into the body of the apical cradle. The exiting suture would initially be introduced through the reel before the apical cradle is fully implanted. Once in place a tool can be used to spin the reel in one direction (i.e., clockwise or counterclockwise) and thereby take up excess length and create tension. Tension is maintained by preventing the reel from rotating in the opposite direction by means of an interference fit design as shown in FIG. 17A (and in an enlarged view in FIG. 17B) or by a ratchet mechanism as shown in FIG. 18A, and in a sectional view in FIG. 18B.

One embodiment of a suture securement device functions by compressing the sutures between two vertical halves of a cylinder. In FIGS. 19A-19D, the halves can separate from each other a short distance to initially allow the suture to pass through. The exterior surface of the halves have a spiral shape such that there is a minimum diameter in one region which increases gradually to a maximum diameter in another region. The halves are surrounded by a rotatable housing. The internal diameter of the housing has raised regions that may contact the exterior surface of the halves. The housing can be rotated relative to halves such that in one position the raised regions correspond to the minimum diameter of the halves and no inward compression of the halves is created, and another position in which the raised region is in apposition with the maximum diameter of the halves, forcing them toward each other and compressing the suture.

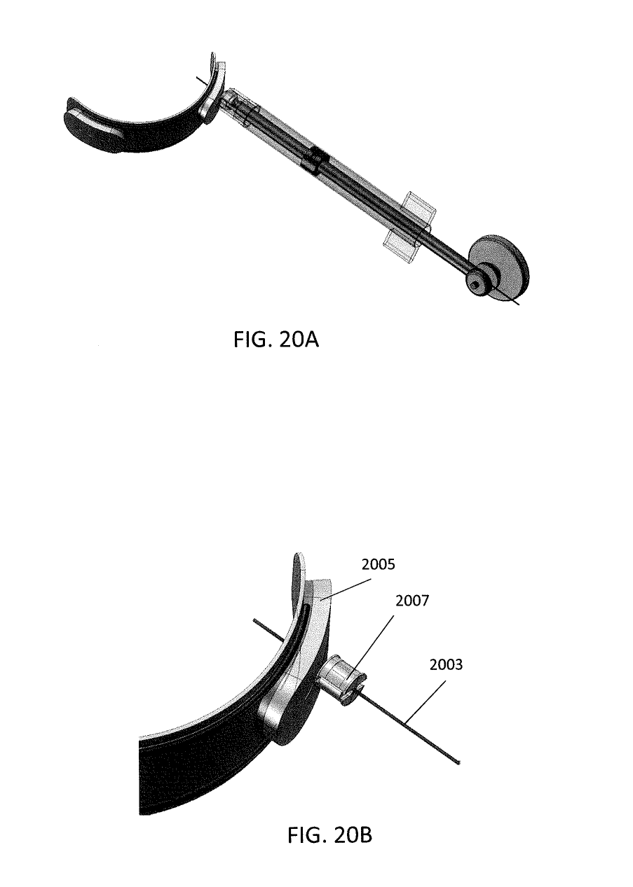

In use, a support chord may be attached to a securement device at a proximal end and passed through the reinforced end region of an apical cradle before or after passing through a heart as described above and illustrated in FIG. 20A. Once attached and anchored to the mitral annuls, the distal end of the support chord 2003 may be anchored by the securement device 2007 against or to the apical cradle, including at the reinforced end 2005, as shown in FIG. 20B.

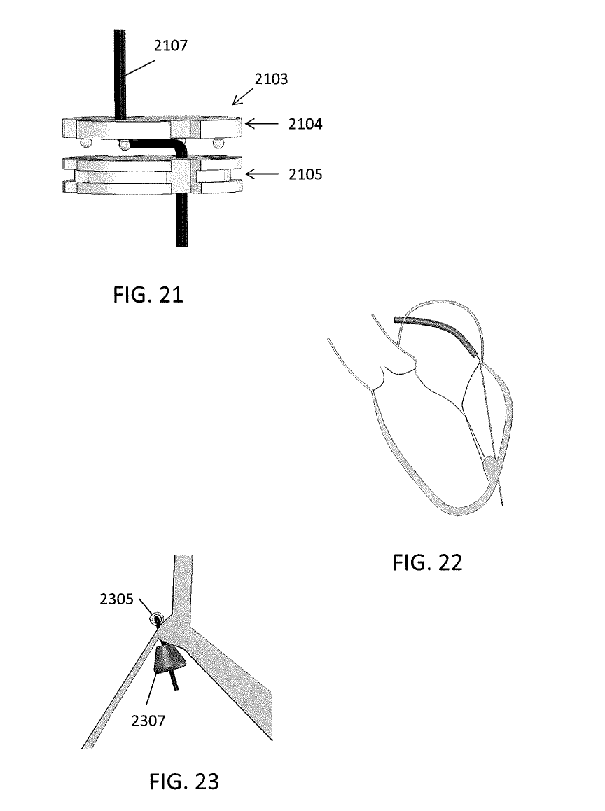

FIG. 21 shows another embodiment of the external chordal securement device 2103 consists of a pair of parallel rigid discs 2104, 2105. Each disc contains a hole through which the chord 2107 can be passed through. The axes of the two holes are offset such that the suture must bend when passing from the one disc to the other. The two discs can then be pulled toward each other and mechanically secured with a snap-fit mechanism, which traps and secures the suture length between them.

Securement may also be achieved by passed the suture between male and female threaded components. When the components are tightened down against each other, the suture is trapped and secured in between.

This invention for atraumatic entry into the left ventricle and mitral annulus anchoring can also be useful for enabling other cardiac technologies. In one example, the invention can be used in conjunction with technologies that enter the left atrium via a transeptal puncture and require a means of identifying and anchoring to the mitral annulus, as shown in FIG. 22. The current invention may couple with a delivery catheter, for example, in the left atrium by mechanical means or a magnetic coupling. For example, a guide wire may be passed from the delivery sheath into a transeptal delivery catheter to provide a guiding path directly to the mitral annulus.

The invention can also be used to create an anchor for prosthetic devices that are delivered to the mitral or aortic annulus, as shown in FIG. 23. The anchor 2305 can be placed through the atrial flange or cuff 2307 of a catheter-based mitral prosthesis, for example, and secure it against the tissue surface. If the chord through the left ventricle is not necessary or desired in this situation, a suture lock can be delivered up along the suture, against the ventricular surface of the annulus and the excess suture can be cut and removed.

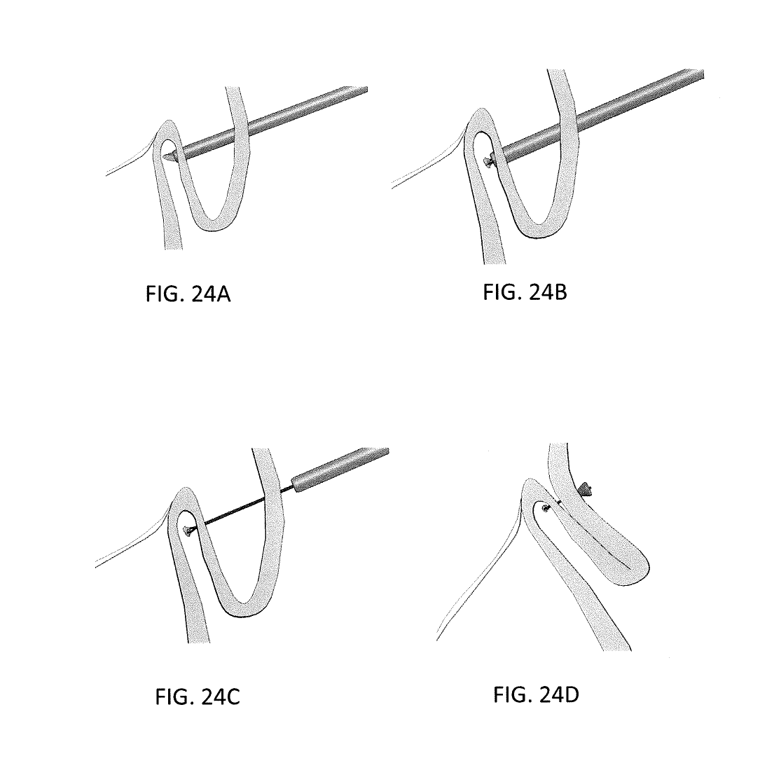

The invention can also be used to create compression and closure of the left atrial appendage (LAA), as illustrated in FIGS. 24A-24D. In patients with atrial fibrillation, blood can pool or stagnate in the LAA leading to clot formation. Closing off or otherwise blocking blood from entering the LAA can help avoid clot formation. As described above, an anchor can be delivered through a small, atraumatic delivery sheath. In this embodiment the sheath and needle puncture the proximal side of the LAA and are passed through to the distal side. The anchor is deployed on the distal side and the sheath is pulled back, leaving the tensioning chord. A suture anchor is then pushed over the tensioning chord to create compression between it and the anchor, thereby compressing the LAA. The anchor would be of a length corresponding to the size of the LAA to ensure complete occlusion. Similarly, a compression pad may also be used on the proximal size to distribute force and increase the area that is compressed.

The process of closing off an undesired space described above can also be beneficial in reducing or eliminating leakage past the cuff of a valve prosthesis (paravalvular leakage). Placing an anchor at a specific site of poor apposition between a valve and the native tissue and pulling and securing tissue against the valve can close gaps that would otherwise cause leakage.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system for reshaping the geometry of a diseased heart, the system comprising:
a cradle for supporting an apical portion of the heart, the cradle having a central portion for supporting the apical portion of the heart, a first end for supporting a first side of the heart, and a second end for supporting a second side of the heart, the cradle having a first reinforced structure disposed on the first end and a second reinforced structure disposed on the second end, wherein the cradle is made from a flexible material;
a first support chord having a distal end and a proximal end, the distal end of the first support chord comprising a first anchor, the proximal end of the first support chord configured to be secured to the first reinforced structure; and
a second support chord having a distal end and a proximal end, the distal end of the second support chord comprising a second anchor, the proximal end of the second support chord configured to be secured to the second reinforced structure.

2. The system of claim 1, wherein the first anchor is configured to be oriented parallel to the first support chord during insertion into the heart and substantially perpendicular to the first support chord after implantation in the heart.

3. The system of claim 1, wherein both the first anchor and the second anchor have a cross-sectional profile sized for allowing passage of the first anchor and the second anchor through an 18 Gauge or smaller needle or a 5 French or smaller sheath.

4. The system of claim 1, wherein first anchor and the second anchor have a length between about 2 to 10 mm.

5. The system of claim 1, further comprising a delivery sheath or needle configured receive the first support chord and the first anchor.

6. The system of claim 1, wherein the first anchor comprises a rigid, elongate tubular member through which the first support chord is passed.

7. The system of claim 6, wherein the tubular member has a cutout or slot extending from both ends of the tubular member.

8. The system of claim 6, wherein the tubular member has two or more holes configured to receive the first support chord.

9. The system of claim 1, wherein the first support chord and the second support chord are made of a flexible material.

10. The system of claim 1, wherein the first support chord and the second support chord are made of a rigid material.

11. The system of claim 1, wherein the first support chord and the first anchor are made from a single shape memory wire or tube having a distal end that is shape set to form the first anchor.

12. The system of claim 1, wherein the cradle is made of a fabric or membrane.

13. The system of claim 1, wherein the cradle comprises one or more bands or struts of shape memory material that are configured to adopt a predetermined shape after insertion.

14. The system of claim 13, wherein the predetermined shape corresponds to an apical portion of the heart.

15. The system of claim 1, wherein the first reinforced structure comprises a pad.

16. The system of claim 15, wherein the pad comprises a layer of semi-rigid material and a layer of compliant material.

17. The system of claim 1, further comprising a first securement device configured to secure the first support cord to the first reinforced structure, wherein the first securement device is configured to adjust the length of the support cord after it has been secured to the first reinforcement structure.

18. The system of claim 17, wherein the first securement device is a spring-loaded or snap-fit clip.

19. The system of claim 17, wherein the first securement device comprises a pair of vertical halves of a cylinder that are surround by a rotatable housing configured to compress the pair of vertical halves together.

20. The system of claim 17, wherein the first securement device comprises a first rigid plate with a first hole for receiving the first support cord and a second rigid plate with a second hole for receiving the first support cord, wherein the first hole and second hole are offset from each other.

21. The system of claim 17, wherein the first securement device comprises a male threaded component and a female thread component.

22. The system of claim 1, wherein the cradle further comprises a pair of rotatable reel mechanisms configured to secure and tighten the first support chord and the second support chord.

23. The system of claim 1, further comprising a cinching device configured to be slidably disposed over both the first support chord and the second support chord in order to reduce the distance between the first support chord to the second support chord.

24. The system of claim 1, wherein the first securement device has one or more holes or slots for receiving the first support chord.

25. A method for reshaping the geometry of a diseased heart, the method comprising:
inserting a first support chord through a first apical portion of the heart;
anchoring the first support chord to a first location on the mitral annulus;
inserting a second support chord through a second apical portion of the heart;
anchoring the second support chord to a second location on the mitral annulus;
placing a cradle against the apical portion of the heart;
securing the first support chord and the second support chord to the cradle; and
tensioning the first support chord and the second support chord to secure the cradle against the apical portion of the heart.

26. The method of claim 25, further comprising:
inserting a sheath and trocar through the first apical portion of the heart to the first location on the mitral annulus;
inserting the sheath and trocar through the mitral annulus and into the left atrium at the first location on the mitral annulus;
removing the trocar from the sheath; and
inserting the first support chord through the sheath.

27. The method of claim 25, wherein tensioning the first support chord and the second support chord reduces the size of the mitral annulus and shortens the length of the left ventricle of the heart.

28. The method of claim 25, wherein the first location on the mitral annulus is opposite the second location on the mitral annulus.

29. The method of claim 25, further comprising inserting the first support chord through papillary muscles in the left ventricle of the heart.

30. The method of claim 25, wherein the first support cord and the second support chord are inserted in a crossing configuration.

31. The method of claim 25, wherein the first support cord and the second support chord are inserted in a non-crossing configuration.

32. The method of claim 25, further comprising tensioning the first support chord and the second support chord laterally inwards by cinching the first support chord and the second support chord together.

33. The method of claim 25, wherein the first location on the mitral annulus is located on an anterior portion of the mitral annulus and the second location on the mitral annulus is located on a posterior portion of the mitral annulus.

34. The method of claim 25, wherein the first support chord is not parallel to the second support chord after insertion into the heart.

35. The method of claim 25, further comprising tensioning the first support chord and the second support chord under echocardiogram visualization until a desired reduction in mitral insufficiency is observed or a desired shortening in the length of the ventricle is observed or a desired change in shape of the mitral annulus is observed.
